(19) United States
(12) Patent Application Publication
(10) Pub. No.: US 2023/0190848 A1
(43) Pub. Date: Jun. 22, 2023

(54) P2ET REDUCES COVID SEVERITY BY INHIBITION OF VIRAL REPLICATION, REDUCTION OF PULMONAR FIBROSIS MARKERS AND MODULATION OF INMUNE RESPONSE

(71) Applicants: PONTIFICIA UNIVERSIDAD JAVERIANA, Bogotá (CO); HOSPITAL UNIVERSITARIO SAN IGNACIO, Bogotá (CO); DREEMBIO S.A., Bogotá (CO)

(72) Inventors: Susana FIORENTINO GÓMEZ, Bogota (CO); Claudia Patricia URUEÑA PINZÓN, Bogotá (CO); Alfonso BARRETO PRIETO, Bogotá (CO); Karol PRIETO SARMIENTO, Bogotá (CO); Alejandra GOMEZ CADENA, Bogotá (CO); Ricardo BALLESTEROS-RAMIREZ, Bogotá (CO); Angel Alberto GARCIA PENA, Bogotá (CO); Pablo ASCHNER, Bogotá (CO); Cindy Mayerly AREVALO OLAYA, Bogotá (CO); Paola Veronica LASSO APRAEZ, Bogotá (CO)

(21) Appl. No.: 18/069,902

(22) Filed: Dec. 21, 2022

Related U.S. Application Data
(60) Provisional application No. 63/292,444, filed on Dec. 22, 2021.

(51) Int. Cl. A61K 36/48; A61K 31/192; A61K 31/216

(52) U.S. Cl. CPC A61K 36/48; A61K 31/192; A61K 31/216; A61K 2236/39; A61K 2236/31

(57) ABSTRACT

The present invention provides compositions and methods for treating or prevent SARS-CoV-2 infections by delivering a therapeutically effective amount of a standardized P2Et extract enriched in polyphenol compounds from Caesalpinia spinosa that inhibits the production of coronavirus particles, reduces pulmonary fibrosis markers, down-regulates pro-inflammatory cytokines, and increases CD3+TL and CD3+CD4−CD8− T Lymphocytes subsets in COVID-19 patients. The disclosed compositions for treating or prevent SARS-CoV-2 infections comprising a standardized P2Et extract enriched in polyphenol compounds from Caesalpinia spinosa that may comprise one or more compounds selected from galloyl quinic acid and gallic acid derivatives and pharmaceutically acceptable excipients.

### CROSS REFERENCE TO RELATED APPLICATIONS

### Field of the Invention

The present invention relates generally to a composition comprising galloyl quinic acid derivatives and methods for treating or prevent SARS-CoV-2 infections comprising administering to the subject a pharmaceutical composition containing a therapeutically effective amount of galloyl quinic acid derivatives and pharmaceutically acceptable excipients.

### Background of the Invention

Severe acute respiratory syndrome caused by Coronavirus 2 (SARS-CoV-2) emerged in December 2019 and was initially identified in Wuhan, Hubei Province of China. It is characterized by its high rate of transmission, mainly through respiratory droplets and contact, high infectivity and ability to cause unusual outbreaks of viral pneumonia. It caused the pandemic of the last century associated with the progression of acute respiratory disease, called Coronavirus Disease 2019 (COVID-19), far exceeding the effects caused by other coronaviruses such as MERS-CoV, of zoonotic origin, which was initially detected in 2012 in Saudi Arabia.

Acute respiratory syndrome is highly transmissible and pathogenic; therefore, it represents a major challenge for humanity because it cause a disease prevalent worldwide with 123 million confirmed cases and 2.70 million deaths (Dong, et al. 2020). The most accepted transmission pathway is associated with the respiratory tract, by person-to-person contact, with an incubation period between 1 to 14 days. Symptoms associated with COVID-19 range from mild upper respiratory tract symptoms to severe acute respiratory distress syndrome. In most cases, patients present with a clinical picture associated with an upper respiratory tract infection, self-limited, with symptoms dissimilar according to the risk group and with rapid progression that evolves rapidly to severe pneumonia and multi-organ failure, usually with fatal consequences in elderly patients with comorbidities such as hypertension, obesity and diabetes (Wern H. et al. 2021).

The disease presents a predominant clinical picture related to: fever, dry cough, dyspnea, anosmia, myalgia, headache, fatigue and use of accessory muscles (scalenes, sternocleidomastoid and intercostal), with predominance of bilateral pulmonary involvement. In some patients, skin manifestations such as erythematous rash, urticaria and varicelliform vesicles associated with alterations in coagulation occur (Zang, Y. et al. 2020). Other findings are decrease in blood lymphocytes, high ferritin index and radiographic evidence of pneumonia, with atypical infiltrate due to diffuse alveolar damage, interstitial inflammation associated with thromboembolism, with involvement in various organs and the vascular endothelium.

Coronavirus 2 (SARS-CoV-2) is a single-stranded, positive-sense RNA virus that has 29 to 881 ribonucleotides that encode 9860 amino acids. The viral genome encodes at least 16 non-structural proteins and the genes that encode the most important structural proteins are S (spicule), E (envelope), M (membrane) and N (nucleocapsid) (Zang J. et al. 2021). Its main component is glycoprotein S, which comprises an RBD domain by which it recognizes the cellular receptor that allows the virus to fuse with the membrane. The S protein is composed of two subunits: S1 and S2; the former contains an RBD receptor binding domain that binds to the host receptor, the latter is involved in the fusion of the viral and cell membrane. The N protein binds throughout the viral genome and takes the form of a curl, this phosphorylated protein has regulatory functions. M is the most abundant protein that provides structural support and E is an essential protein for the assembly and release of virions.

In relation to the virus receptor, the angiotensin-converting enzyme type 2 (ACE2) has been identified as the main receptor for viral entry, as well as the transmembrane serine protease receptor 2 (TMPRSS2), which is a critical regulator essential for the entry of SARS-CoV-2 into the cell (Bilinska, K. et al. 2020). Cells with high levels of ACE2 and TMPRSS2 expression exhibit a strong binding capacity to the virus and are particularly susceptible to infection. Once the SARS-CoV-2 virus invades the human host, the first step is to neutralize its replication and spread and the first receptor that attaches the virus to the membrane of mucosal epithelial cells in the nose, trachea and alveolar cells type 2 (AT2) is represented by ACE2 and employs TMPRSS2 involved in contact with the virus S protein leading to endosomal fusion within the cell, release of viral ssRNA and binding to recognition receptors. Once AT2 cells are infected, an innate immune response is generated. With the entry of the virus numerous virions occur at the level of the respiratory tract along with the spread of the virus towards the lung tissue. SARS-CoV-2 infection is capable of producing an excessive immune reaction.

Innate lymphoid cells (ILCs) are mainly concentrated on barrier surfaces such as the skin, intestine, and lungs, regulate tissue homeostasis and rapidly generate a response to environmental signals, their main function is to initiate, shape and refine the immune response through the production of cytokines. They are currently recognized as the innate counterpart of cooperating CD4+ T cells, and are divided into ILC1, ILC2, and ILC3.

In the lungs, the subpopulation of ILC2 cells limits virus and allergen-induced type 2 response via production of type 2 cytokines such as interleukin IL-4, IL-5, IL-9, and IL-13 that promote eosinophil recruitment, completion of inflammatory response, and tissue repair. Thus, ILC2 cells retain lung integrity and play a key role in the defense of the host against viral infections, however, an aberrant activity of ILC2 in the lung triggers a severe inflammation of the respiratory tract.

Innate immune cells, mostly polymorphonuclear (PMN) cells, ILC especially ILC2, NK cells and others, are recruited by cytokines and chemokines released during early response by antigen-presenting cells, further amplifying the immune response, using TLRs and promoting cytokine bursting. Among them, innate NK cells in their different subtypes are of crucial physiological importance because they are enriched with TLR7 (and other TLRs) and produce type I IFNs. In SARS-CoV-2 infection, the population of circulating NK cells migrates from the peripheral blood to the lungs (Zheng, et al 2020) and their importance in eliminating viral infection has been recognized, supported by observations in patients with NK cell deficiencies who are predisposed to recurrent viral infections. Their role in the response of T cells and in the death of infected cells is critical (Kumar A., et al. 2021).

SARS-CoV-2 infection mainly affects and causes more serious disease in the elderly. NK cells have been recognized to have greater numbers during childhood and progressively decrease with aging. It also happens with neutrophils and with the number and function of lymphocytes that decrease with aging, this situation is evident in adult patients admitted to intensive care units (ICU) and who suffer from a greater number of comorbidities and risks of presenting adverse outcomes (Wang et al., 2020). Meta-analyses of comorbidities suggest that hypertension as well as diabetes, cardiovascular and respiratory disease are prevalent in adult patients admitted to ICU; those conditions increase susceptibility to the virus and are related to the pathogenesis of COVID-19.

Another important aspect to consider has to do with the fact that chronic diseases share several characteristics with the infectious state caused by viral agents, such as the proinflammatory state and the attenuation of the innate immune response (Yang, J. et al. 2020).

Moreover, during the SARS-CoV-2 attack, a storm of pro-inflammatory cytokines and excessive activation of monocytes and macrophages responsible for producing the majority of oxidants, characteristic in the damage due to acute lung disease, are triggered. There is evidence of a relationship between the generation of pro-inflammatory elements and the production of reactive oxygen species (ROS) in the progression of lung disease caused by coronavirus (Derouiche, 2020), due to the cooperation between the functions of cytokines, chemokines and adhesion molecules that control the inflammatory response in the lungs.

Idiopathic pulmonary fibrosis (IPF) is also considered a sequel to infection caused by SARS-CoV-2 ranging from fibrosis associated with pneumonia to lung injury. Acute exacerbations of IPF present evidence in the triggering of viral infection, including coronavirus infection (Spangnolo, P. et al. 2020). IPF shares with severe COVID-19 infection the main risk factors, including: age, male gender and the presence of comorbidities such as hypertension and diabetes. Studies relate the presence of type 2 cytokines such as IL-13 within the profibrotic factors present in bronchoalveolar fluid (van der Ploeg. E. K., et al. 2018), for this reason, the efforts in ICU patients focus on administering the authorized antifibrotic treatments, such as the case of pirfenidone and nintedanib, which differ in their mechanism of action and effectiveness to attenuate the decline in lung function.

Other synthetic drugs identified as potential inhibitors of SARS-CoV-2 replication are remdesivir, favipiravir, rivabirine and galidesivir.

In relation to the use of natural products for the management of inflammation as a clinical manifestation in diseases such as cancer, diabetes and respiratory diseases, extracts of Echinacea spp, Sambucus nigra, Pelargonium sidoides and Curcuma longa, among others, have been evaluated, the ability to reduce the expression of transcription factors related to proinflammatory cytokines is attributed to them. Recently, chloroquine and hydroxychloroquine and their relationship with the quina tree suggested that quinine could be useful to treat COVID-19 disease, without clinical evidence on its effects to date, in contrast, cardiac and renal complications have been detected.

Regarding natural products and extracts for the treatment of infection caused by SARS-CoV-2, the following references are found in the patent literature:

Patent US2021/0236529 discloses the use of hydrolysable tannins to prevent and mitigate acute respiratory syndrome associated with viral and microbial diseases, especially in coronavirus-caused infections such as SARS-CoV-2 in humans. The publication presents a method of preventing, inhibiting, or treating COVID-19 by administering one or more hydrolyzable tannins selected from pentagaloyl glucose, chebulinic acid, chebulagic acid, pedunculagin, telimagrandin I and II, geranin, corilagin, casuaricitin, and nufarin where tannins are administered after exposure to the virus in a patient with acute respiratory distress syndrome symptoms.

Application US2021/0236580 provides a method of treating or inhibiting the symptoms of acute respiratory syndrome, especially associated with COVID-19 viral infection comprising administering by conventional methods an amount of a natural extract to modulate the innate immune response and cytokine production. The extract comprises a series of metabolites and their combinations capable of modulating the production of cytokines and the innate immune response, made from the fruit of Terminalia chebula, Terminalia bellerica, Terminalia arjuna, Emblica officinalis, in patients with symptoms such as hyperinflammation. The method allows down-regulation of IL-6 and IL-8 and downstream gene regulation and maintains the levels of IL-12, IFN-α and IFN-γ so that an immune response against SARS-CoV-2 is produced by administering an extract enriched in at least 40% by weight in tannins.

A method for the treatment and prevention of COVID-19 is presented in US2021/0361700 by administering a combination of natural compounds selected from the group comprising: zinc, quercetin, vitamin E, and epigallocatechin gallate (EGCG) to reduce disease severity and improve the immune response to vaccination with biological COVID-19 vaccines. The disclosure shows that EGCG down-regulates the production of pro-inflammatory cytokines such as TNF-α, IL-6 and IL-8 in acute viral respiratory infection; thus, it evidences a significant reduction in cytokine storm and lung injury. In addition, it suppresses inflammatory mediators, TLR4, NF-κB and ROS production according to preclinical evidence. The method succeeds in inhibiting the entry of the virus into cells or their replication, increases T cell function, reduces inflammation, cytokine levels, ROS and tissue damage.

US2021/0393580 discloses a composition that includes procyanidins selected from the group of: δ-(3,4-dihydroxyphenyl)-φ-valerolactone, δ-(3-methoxy-4-hydroxy-phenyl)-γ-valerolactone, catechin, epicatechin, ferulic acid, gallic acid, 4-hydroxybenzoic acid, caffeic acid, taxifoline, and protocatechic acid or combinations thereof, for preventing or treating endothelial inflammation—endothelitis—and/or endothelial systemic dysfunction caused by coronavirus disease (COVID-19), including treatment of post-COVID-19 symptoms, as a result of an alteration in vascular systemic microcirculatory function. The peroral composition comprises procyanidins from an extract of pine bark, grape seed, apple, cocoa, peanut bark, blueberries or combinations thereof with at least one excipient, in a dose ranging from 24 mg to 300 mg per day.

Furthermore, US2021/0393679 discloses a molecular complex comprising EGCG-Zn⁺²(epigallocatechin-zinc gallate) that exhibits high affinity alone or through its individual species with the SARS-CoV-2 virus and its molecular markers, thereby succeeding in suppressing viral activity at a level greater than 99%. The EGCG-Zn⁺² complex exhibits low toxicity in transfected human cells and better pharmacokinetic parameters than the EGCG and Zn⁺² combination in humans. The document discloses a method of improving the chemoprophylaxis or treatment of viral disease to minimize tissue damage. The format complex exhibits significant synergistic activity in viral inhibition when administered for periods ranging from 1 to 3, preferably 1 to 10, and particularly 1 to 30 days. Methods for obtaining the structure of SARS-CoV-2 proteins that recognize the EGCG-Zn⁺² complex by molecular coupling and identify the polypeptides with which the EGCG-Zn⁺² complex chemically interacts are mentioned in the document: PLP, RdRp, ACE2-RBD, 3CLpro, NSP15 at active and alternative site, and spike protein S. The complex reduces the biological activity of SARS-CoV-2 by blocking viral translation and transfection.

US2022/0031654 discloses a composition comprising bioflavonoids for re-establishing the regulation and homeostasis of defense mechanisms against a disease caused by SARS-Cov-2. The composition comprises a standardized bioflavonoid extract enriched with at least one B-ring free flavonoid and with flavan in the range of 1 to 98% w/w, the standardized and enriched B-ring free extract is obtained from the root of Scutellaria baicalensis and the flavan is obtained from the woody part or from the heartwood of Acacia catechu. The B-ring free flavonoid comprises at least one flavonoid selected from the group comprising: baicalin, baicalein, baicalein glycoside, wogonin and wogonin glucoronide, wogonin glycoside, oroxylin, oroxylin glycoside, oroxylin glucoronide, chrysin, chrysin glycoside, chrysin glucoronide, esctelarin, esctelarin glycoside, norwogonine, norwogonine glycoside and galangin, or combinations thereof. And wherein the standardized flavan-enriched bioflavonoid extract comprises catechin, epicatechin, catechinatechinate, galocatechin, epigallocatechin, EGCG, epitheflavin, galocatechinatechinate, theaflavin, theaflavin gallate, or combinations thereof.

Other studies in silico, related to the ability of tannins to inhibit the binding capacity of the SARS-Cov-2 virus, concentrate on evaluating the 3-chymotrypsin-cysteine protease 3CL^{Pro} enzyme of the virus involved in its replication (Khalifa I. et al. 2020). This study evaluates 19 hydrolysable tannins that recognize 3CL^{Pro} catalytic dyad residues that use a MOE09 molecular coupling operating environment, specifically: castalin, bicornin, grandinin, tercatain, granatin A, tellimagradin I, geraniin, casuarinin, strictinin, pedunculagin, punicalin, chebulagic acid, casuarictin, β-pedunculagin, potentillin, isoterchebin, roxbin B, repandusinic acid A, and terchebin. The results suggest that the natural metabolites pedunculagin, tercatain and castalin manage to form a complex of recognition and binding toSARS-CoV-2-3CL ^{pro}, stable and without fluctuations so they can function as possible inhibitors against SARS-Cov-2.

Thi Thanh Hanh, N. et al. (2021) disclose the relationship structure activity of some plant-derived polyphenols against SARS-CoV-1 M^{pro}, as nutraceuticals that improve the health of COVID-19 patients. The study evaluates the ability of tannic acid and its combinations with myricetin, puerarin and daidzein on the activity of the M viral protein. In particular, they evaluate the ability of black garlic and 15 polyphenols to inhibit the activity of M^{pro} with IC₅₀ values ranging from 137 μg/mL and 9 to 197 μm, respectively. The structure activity ratio of the polyphenols is based on the interaction of the hydroxyl groups present at positions C3″, C4″, C5″ of ring B, C3 of ring C, C7 of ring A and the double bonds present between carbons 2 and 3 of ring C, as well as the glycosylation present at carbon 8 of ring A which contribute to the inhibitory activity of flavonoids on M^{pro}.

Alossaimi M. A., et al. 2022 evaluated the activity of Pelargonium sidoides root extract native to South Africa, a Geraniaceous plant, used locally to treat symptoms related to respiratory tract infections, including tuberculosis. The study evaluated the activity of the most representative biomolecules, including one phenolic acid, one flavan derivative and two coumarins (scopoletin and umclalin). The results of in vitro studies against SARS-CoV-2 suggest potential antiviral activity and selectivity. And molecular coupling studies suggest that biomolecules interfere in the interaction between SARS-CoV-2 S-speculum protein and the ACE2 receptor.

In the state of the art, there is a persistent need to find safe and effective phytotherapeutic alternatives for the management of viral infection caused by SARS-CoV-2 that help in the treatment of acute respiratory disease, inhibit the replication of the virus, regulate the homeostasis of hematopoietic cells, modulate the response of the innate immune system and downregulate the eosinophil count and the production of pro-inflammatory cytokines, thereby preventing the progression of the disease to the most serious adverse events due to possible pulmonary fibrosis.

The studies reported the state of the art concentrate on evaluating the activity of isolated biomolecules from natural extracts whose structure differs from the polyphenols present in the extract of the pod of the Caesalpinia spinosa plant, called P2Et, a plant collected in Villa de Leyva, Boyacá, Colombia and identified by Luis Carlos Jimenez of the Colombian National Herbarium (species number COL523714) with a contract for access to genetic resources and their derived products granted by the Ministry of the Environment in Colombia.

The metabolites identified in the standardized extract P2Et are a variety of polyphenols derived from galoyl-quinic acid with high antioxidant activity that decrease lipid peroxidation and tissue damage and induce complete autophagy in tumor or stressed cells (Prieto K., et al., 2019; Sandoval, T. A., et al., 2016). In addition, P2Et decreases proinflammatory cytokines dependent on NFkB activation, such as IL-6 (Urueña C., et al., 2013). In the present disclosure, the standardized P2Et extract inhibits the production of viral particles in infected cells, reduces oxidative stress and maintains the expression of danger signals during viral infection, and further reduces inflammation and progression of pulmonary fibrosis in vivo mechanisms of action not reported for polyphenols by another state-of-the-art research.

The combination of galloyl quinic acid and gallic acid derivatives in the standardized P2Et extract, present in the compositions of the invention were obtained through a method to extract the C. spinosa with ethanol (DER 20-33,3:1) and subsequently fractionate it with ethyl acetate. Previously, the P2Et extract was standardized using HPLC-RP and HPLC/MS/UV techniques, as well as isolation of the different identified components (Sandoval, T. A., et al., 2016), to demonstrate its therapeutic activity and ensure batch-by-batch consistency by quality assurance of cultivation, harvesting and manufacturing processes.

### SUMMARY OF THE INVENTION

Firstly, the disclosure relates to a therapeutical composition comprising a standardized P2Et extract enriched in polyphenol compounds from Caesalpinia spinosa that inhibits the production of coronavirus particles, reduces pulmonary fibrosis markers, down-regulates pro-inflammatory cytokines, and increases CD3+TL and CD3+CD4−CD8− T Lymphocytes subsets in COVID-19 patients.

Secondly, the disclosure is directed to a therapeutical composition comprising a standardized P2Et extract including one or more compounds selected from galloyl quinic acid and gallic acid derivatives that inhibits the production of coronavirus particles, reduces pulmonary fibrosis markers, down-regulates pro-inflammatory cytokines and increases CD3+TL and CD3+CD4−CD8− T Lymphocytes subsets in COVID-19 patients.

Thirdly, the disclosure is directed to a method for treating or preventing SARS-CoV-2 infections by delivering a pharmaceutical composition to a subject in need thereof, comprising administering to said subject a pharmaceutical composition that contains a therapeutically effective amount of standardized P2Et extract enriched in polyphenol compounds from Caesalpinia spinosa and pharmaceutically acceptable excipients.

Moreover, the disclosure is directed to a method that inhibits the production of coronavirus particles, to reduce pulmonary fibrosis markers, down-regulate pro-inflammatory cytokines and increase CD3+TL and CD3+CD4−CD8− T Lymphocytes subsets in a subject suffering from SARS-CoV-2 infections comprising: administering to a subject in need thereof a pharmaceutical composition that contains a therapeutically effective amount of standardized P2Et extract enriched in polyphenol compounds from Caesalpinia spinosa and pharmaceutically acceptable excipients.

Furthermore, the disclosure is directed to a method to inhibit the production of coronavirus particles in a subject infected with coronavirus comprising administering a pharmaceutical composition that contains a therapeutically effective amount of standardized P2Et extract enriched in polyphenol compounds from Caesalpinia spinosa.

Furthermore, the disclosure is directed to a method of reducing pulmonary fibrosis markers of a subject infected with coronavirus comprising administering a pharmaceutical composition containing a therapeutically effective amount of standardized P2Et extract enriched in polyphenol compounds from Caesalpinia spinosa.

In addition, the disclosure is directed to a method for normalizing the cytokine profile of a subject infected with coronavirus comprising administering a pharmaceutical composition containing a therapeutically effective amount of standardized P2Et extract enriched in polyphenol compounds from Caesalpinia spinosa.

Finally, the disclosure is directed to a method to facilitate normalization of the CD3+TL and CD3+CD4−CD8− T Lymphocytes subsets in the subject infected with coronavirus, comprising administering a pharmaceutical composition containing a therapeutically effective amount of standardized P2Et extract enriched in polyphenol compounds from Caesalpinia spinosa.

### BRIEF DESCRIPTION OF THE FIGURES

FIG. 1 shows the standardized P2Et extract inhibition of the infectious viral particles production in L929 infected cells.

FIG. 2 shows the standardized P2Et extract effect on intracellular ROS in fibroblast L929 infected with MHV-A59 virus.

FIG. 3 shows the calreticulin expression associated to virus infection of fibroblasts L929 treated with the standardized P2Et extract of the invention.

FIG. 4 shows the standardized P2Et extract effect on progression of bleomycin-induced fibrosis mice model. P2Et extract in mice lungs reduces: inflammatory infiltrate cells (4A), innate cells infiltrate (4B), ILC2 subset infiltrate (4C) and eosinophils infiltrate (4D). P2Et decreases soluble factors associated to pulmonary fibrosis progression: IL-22 (4E), TGFb and TNFa receptor (4F).

FIG. 5 shows the hospital discharge for the P2Et group and placebo in patients with COVID19.

FIG. 6 shows the P2Et modulation of patients' pro-inflammatory cytokines: IL-5 (FIG. 6A), IL-6 (FIG. 6B), G-CSF (FIG. 6C), IL-10 (FIG. 6D), IL-18 (FIG. 6E), IFN-γ (FIG. 6F), MCP-2 (FIG. 6G) and MCP-1 (FIG. 6H).

FIG. 7 shows the frequency increase of CD3+T lymphocytes and CD3+CD4−CD8− T lymphocytes subsets in the P2Et group of COVID-19 patients′.

FIG. 8 shows the total ILCs, ILC2 and NK subsets in healthy donors or COVID patients after treatment with the standardized P2Et extract or placebo.

### DETAILED DESCRIPTION OF THE INVENTION

To facilitate the description of the components of the present disclosure, the following definitions are provided for the terms used in the specifications.

As used herein, the term “treatment” refers to medical management of a disease, disorder, or condition of a subject (e.g., a human or non-human mammal, such as a primate, horse, cat, dog, goat, mouse, or rat). In general, an appropriate dose or treatment regimen comprising a pharmaceutical composition containing the standardized P2Et extract enriched in polyphenol compounds from Caesalpinia spinosa of the present disclosure is administered in an amount sufficient to elicit a therapeutic effect or therapeutic benefit. Therapeutic effect or therapeutic benefit includes improved clinical outcome; modulation of immune response to normalize inflammatory cytokine activity; alleviation of symptoms associated with a disease; decreased occurrence of symptoms; improved quality of life; longer disease-free status; diminishment of extent of disease, stabilization of disease state; delay of disease progression; remission; survival; prolonged survival; or any combination thereof.

As used herein, the term “prevent” refers to a prophylactic treatment of a disease i.e. SARS-CoV-2 infection, which include a treatment administered to a subject who does not exhibit signs of a disease or exhibits only early signs, for the purpose of decreasing the risk of developing the infection or further progression of the disease. A prophylactic treatment can mean preventing recurrence of a disease or condition in a patient that has previously been treated for the disease or condition, e.g., by preventing relapse or recurrence of SARS-CoV-2 infection.

As used herein, the term “therapeutically effective amount” or “effective amount” of the standardized P2Et extract enriched in polyphenol compounds of this disclosure refers to an amount of one or more compounds selected from galloyl quinic acid and gallic acid derivatives sufficient to result in a therapeutic effect, within a risk/benefit balance that is acceptable for any medical treatment.

As used herein, the term “standardized P2Et extract” of this disclosure refers to a drug substance that complies EMA requirement for “standardized extracts”, it means that identified constituents in P2Et extract are understood to fully account for proven therapeutic activity, as previously shown. Standardized P2Et extract may comprise one or more compounds selected from galloyl quinic acid and gallic acid derivatives.

The term “therapeutical composition enriched in polyphenol compounds” refers to a pharmaceutical composition containing the standardized P2Et extract from Caesalpinia spinosa to be administered in a subject suffering from SARS-CoV-2 infections for the treatment or prevention of the disease. Specifically, a pharmaceutical dosage form containing the standardized P2Et extract with one or more pharmaceutically acceptable excipients for oral administration in solid or liquid pharmaceutical forms as tablets, enteric or conventional capsules, buccal forms and oral liquids, suspensions, or solutions; for topical administration in heterodispersed W/O or O/W forms as creams, gels and ointments and for parenteral or rectal administration.

The term “pharmaceutically acceptable excipient” refers to biologically compatible vehicles, which are described in greater detail herein, that are suitable for administration to a human or other non-human mammalian subject and generally recognized as safe or not causing a serious adverse event.

In a first aspect, the invention relates to a therapeutical composition comprising a standardized P2Et extract enriched in polyphenol compounds from Caesalpinia spinosa that inhibits the production of coronavirus particles, reduces pulmonary fibrosis markers, down-regulates pro-inflammatory cytokines, and increases CD3+TL and CD3+CD4−CD8− T Lymphocytes subsets in COVID-19 patients.

The standardized P2Et extract may comprise one or more compounds selected from galloyl quinic acid and gallic acid derivatives, specifically comprising between 70% and 100%, relative to the total weight of the composition, from one or more compounds selected from n-O-galloylquinic acid, di-O-galloylquinic acid, tri-O-galloylquinic acid, tetra-O-galloylquinic acid, n-O-galloylquinic acid methyl ester, n-O-galloylquinic acid ethyl ester, di-O-galloyl quinic methyl ester, di-O-galloyl quinic ethyl ester, tri-O-galloyl quinic methyl ester, tri-O-galloyl quinic ethyl ester, di-O-galloyl quinic methyl gallate ester, tri-O-galloyl quinic methyl gallate ester, di-O-galloyl quinic ethyl gallate ester, tri-O-galloyl quinic ethyl gallate ester, n-O-galloyl(digalloyl)quinic acid, n-O-galloyl(galloyl)quinic acid, di-O-galloyl(digalloyl)quinic acid or di-O-galloyl(galloyl)quinic acid.

In a specific embodiment, the standardized P2Et extract comprises: (i) between 70% and 95%, relative to the total weight of the composition of one or more compounds selected from: n-O-galloylquinic acid, di-O-galloylquinic acid, tri galloylquinic acid, tetra-O-galloylquinic acid, n-O-galloylquinic acid methyl ester, n-O-galloylquinic acid ethyl ester, di-O-galloyl quinic methyl ester, di-O-galloyl quinic ethyl ester, tri-O-galloyl quinic methyl ester, tri-O-galloyl quinic ethyl ester, di galloyl quinic methyl gallate ester, tri-O-galloyl quinic methyl gallate ester, di galloyl quinic ethyl gallate ester, tri-O-galloyl quinic ethyl gallate ester, n-O-galloyl(digalloyl)quinic acid, n-O-galloyl(galloyl)quinic acid, di-O-galloyl(digalloyl)quinic acid or di-O-galloyl(galloyl)quinic acid. (ii) between 5% and 30%, relative to the total weight of the composition, of one or more compounds selected from: gallic acid, ethyl gallate or methyl gallate.

Said composition may be formulated with one or more pharmaceutically acceptable excipients for oral administration in solid or liquid pharmaceutical forms, for topical administration in heterodispersed forms (W/O creams, O/W creams, gels and ointments) and for parenteral or rectal administration. The compositions of the invention may be administered to humans and other mammals via the oral, rectal, parenteral, topical, intravaginal, buccal route or as a nasal or oral spray.

The compositions for oral administration which contain the standardized P2Et extract enriched in polyphenol compounds include the well-known oral pharmaceutical forms, such as: tablets, capsules, buccal forms and oral liquids, suspensions or solutions. A preferred embodiment comprises solid dosage forms like hard capsules, including conventional or enteric capsules, the latter designed to remain intact in the stomach but dissolve and release the drug substance in the intestine, it means is stable at the highly acidic pH of the stomach, but degrade rapidly at alkaline pH of small intestine.

Capsules may contain mixtures of the drug substance with pharmaceutically acceptable excipients such as: starch, sugars, artificial sweeteners, powdered celluloses (carboxymethylcellulose, methyl cellulose, ethyl cellulose, hydroxypropyl methyl cellulose), flours, gelatins and gums. Likewise, the oral compositions disclosure may be conventional formulations or sustained-release or controlled-release formulations which alter the absorption of the drug substance. Polymer film coatings which may be used for the enteric embodiment include cellulose acetate phthalate, hydroxypropyl methylcellulose phthalate and methyl acrylic acid co-polymers.

In a second aspect, the disclosure is directed to a therapeutical composition comprising a standardized P2Et extract including one or more compounds selected from galloyl quinic acid and gallic acid derivatives that inhibits the production of coronavirus particles, reduces pulmonary fibrosis markers, down-regulates pro-inflammatory cytokines and increases CD3+TL and CD3+CD4−CD8− T Lymphocytes subsets in COVID-19 patients.

Also, the disclosure is directed to a method for treating or preventing SARS-CoV-2 infections by delivering a pharmaceutical composition to a subject in need thereof, comprising administering to said subject a pharmaceutical composition that contains a therapeutically effective amount of standardized P2Et extract enriched in polyphenol compounds from Caesalpinia spinosa and pharmaceutically acceptable excipients.

Moreover, the disclosure is directed to a method that inhibits the production of coronavirus particles, to reduce pulmonary fibrosis markers, down-regulate pro-inflammatory cytokines and increase CD3+TL and CD3+CD4−CD8− T Lymphocytes subsets in a subject suffering from SARS-CoV-2 infections comprising: administering to a subject in need thereof a pharmaceutical composition that contains a therapeutically effective amount of standardized P2Et extract enriched in polyphenol compounds from Caesalpinia spinosa and pharmaceutically acceptable excipients.

This disclosure provides treatment of a subject with mild, moderate, or severe SARS-CoV-2 infections comprising administering to the subject an effective amount of standardized P2Et extract enriched in polyphenol compounds and pharmaceutically acceptable excipients.

The dosage levels of the standardized P2Et extract enriched in polyphenol compounds of the disclosure in the pharmaceutical composition provided may vary to reach the desired therapeutic response depending on the condition, size, weight, body surface area, age, sex, type and severity of the disease, formulation, route of administration, and concomitant drugs administered to the patient, which can be readily determined by a person trained in the skill.

The total daily dose of the standardized P2Et extract enriched in polyphenol compounds of the disclosure in the pharmaceutical composition may vary within the range from about 0.001 to about 1000 mg/kg/day. For oral administration purposes, the preferred doses are within the range from about 0.5 to about 50 mg/kg/day. Effective daily dose may be divided into multiple doses for administration purposes, and consequently the disclosure comprises compositions of single doses that contain the effective amount or multiple doses to reach the effective daily dose after several administrations.

A preferred embodiment is the oral administration for a plurality of times, preferably once, twice or three times daily.

The duration of treatment may vary from about 1 week to about 16 weeks to reach the desired therapeutic response. A preferred embodiment of the method to treat or prevent SARS-CoV-2 infections disclosure comprising: administering to a subject a daily dose from about 0.4 mg to about 4000 mg via oral route from 1 to 28 days, preferably from about 20 mg to about 2000 mg via oral route for one to 28 days.

The scientific facts upon which the present disclosure is based, which should not be understood as limiting the disclosure, are presented hereinbelow for illustrative purposes.

### Plant Material, Extraction and Fractionation

As previously defined, the term “standardized P2Et extract” refers to a drug substance obtained by extraction and fractionation procedures from Caesalpinia spinosa pods under good manufacturing practice (Sandoval, T. A., et al., 2016, incorporated by reference in its entirety). C. spinosa pods were collected in Villa de Leyva, Boyacá, Colombia in March 2007 and identified by Luis Carlos Jiménez from the Colombian National Herbarium (voucher specimen number COL 523714). Briefly, fresh pods were washed and dried in an oven at 45° C. and then ground down to obtain pulverized plant material (Specifications: 40 mesh 50-70%, 60 mesh 15-30%, 80 mesh 5-15%). Quality Control approved pulverized material was extracted with ethanol 96% (DER 20-33,3:1) in a rotary percolator over a period of 6 h at room temperature. The ethanol crude extract was concentrated under vacuum at 40° C. using an evaporator, afterwards, the extract concentrated was mixed with anhydrous colloidal silicon dioxide (ratio 1:1) and dried in an oven at 50° C. Then, the matrix was fractionated with the following solvents: chloroform and ethyl acetate using vacuum tanks. Finally, the ethyl acetate fraction was concentrated under vacuum at 40° C. to obtain the drug substance (yield 5% from pulverized plant material).

### P2ET Chemical Characterization

Standardized P2Et extract is a drug substance that complies EMA requirement for “standardized extracts”, it means that identified constituents in P2Et extract are understood to fully account for proven therapeutic activity. As previously published by our research group, P2Et extract constituents comprise galloyl quinic acid and gallic acid derivatives in a standardized percentage that allow guaranteeing batch-to-batch consistency through quality assurance practices applied to agricultural and manufacturing processes (Sandoval, T. A., et al., 2016, incorporated by reference in its entirety).

Specifically, the standardized P2Et extract may comprise between 70% and 100%, relative to the total weight of the composition, from one or more compounds selected from n-O-galloylquinic acid, di-O-galloylquinic acid, tri-O-galloylquinic acid, tetra-O-galloylquinic acid, n-O-galloylquinic acid methyl ester, n-O-galloylquinic acid ethyl ester, di-O-galloyl quinic methyl ester, di-O-galloyl quinic ethyl ester, tri-O-galloyl quinic methyl ester, tri-O-galloyl quinic ethyl ester, di-O-galloyl quinic methyl gallate ester, tri-O-galloyl quinic methyl gallate ester, di-O-galloyl quinic ethyl gallate ester, tri-O-galloyl quinic ethyl gallate ester, n-O-galloyl(digalloyl)quinic acid, n galloyl(galloyl)quinic acid, di-O-galloyl(digalloyl)quinic acid or di galloyl(galloyl)quinic acid.

More preferably, the standardized P2Et extract comprises: (i) between 70% and 95%, relative to the total weight of the composition of one or more compounds selected from: n-O-galloylquinic acid, di-O-galloylquinic acid, tri-O-galloylquinic acid, tetra-O-galloylquinic acid, n-O-galloylquinic acid methyl ester, n-O-galloylquinic acid ethyl ester, di-O-galloyl quinic methyl ester, di-O-galloyl quinic ethyl ester, tri-O-galloyl quinic methyl ester, tri-O-galloyl quinic ethyl ester, di-O-galloyl quinic methyl gallate ester, tri-O-galloyl quinic methyl gallate ester, di-O-galloyl quinic ethyl gallate ester, tri-O-galloyl quinic ethyl gallate ester, n-O-galloyl(digalloyl)quinic acid, n-O-galloyl(galloyl)quinic acid, di-O-galloyl(digalloyl)quinic acid or di-O-galloyl(galloyl)quinic acid. (ii) between 5% and 30%, relative to the total weight of the composition, of one or more compounds selected from: gallic acid, ethyl gallate or methyl gallate.

### Cell Culture and Virus Production

Virus stocks of Mouse hepatitis virus A59 strain (VR-764) and their producer cell lines NCTC-1469 (CCL-9.1) were obtained from the American Type Culture Collection (Manassas, Va., USA). NCTC1469 were used for propagation of MHV-A59, cell cultures were maintained in Dulbecco's modified Eagle's medium (DMEM) supplemented with 4.5 g/I D-glucose, 2 mM L-glutamine, 100 U/mL penicillin, 100 μg/mL streptomycin and 10% horse serum (GIBCO Laboratories, Grand Island, N.Y.). Propagation of virus stocks was performed in cultures of permissive cells, the cells on T75 flaks were infected with the multiplicity of infection (MOI) of 1.0 or 0.1 for 1 h at 37° C. of virus suspension, replacing it with 10 ml cell culture medium. MHV-A59-infected cells were frozen in their culture flasks after 48 h. They were subjected to three freeze-thaw cycles to allow release of the virus. The contents of the flasks were centrifuged at 3,000 g for 10 min to separate virus from cell debris. The supernatant was passed through a filter having a pore size of 0.20 μm and freezer at −70° C. For titration, L929 cells kindly provided by Carlos Guerrero (Universidad Nacional de Colombia) were seeded in 12-well plates at a concentration of 1.2×105 for well and cultured overnight. Supernatant virus was assayed following serial dilution in DMEM, after removal of the culture medium, for each 10-fold dilution up to 10-5, 12 wells were inoculated with 200 μl of the virus suspension. Following adsorption at room temperature for 60 minutes, plates were overlaid with DMEM plus 2% FBS containing 0.6% agarose. Plaques were visualized in the L929 monolayers at 48 hours post infection with 0.5% crystal violet following fixation with 20% formalin for 30 minutes.

### Example 1. Inhibition of Infectious Viral Particles Production Assay

To determine if the P2Et extract of the invention can inhibit virus infection L929 cells were seeded in 12-well plates at a concentration of 1.2×105 for well and cultured overnight. Cells were pretreated for 2 h with the P2Et or controls, washed twice with PBS 1× and inoculated with 200 μl of the virus suspension to 0.1, 0.01 or 0.001 MOI in DMEM, 1 h at room temperature in mechanical agitation. The virus inoculum was removed, the cells were treated again with extracts for 6, 12 and 24 h. Supernatants were collected for qPCR and plaque assays. The cells were collected for evaluation of proteins by western blot and ROS assays by flow cytometry.

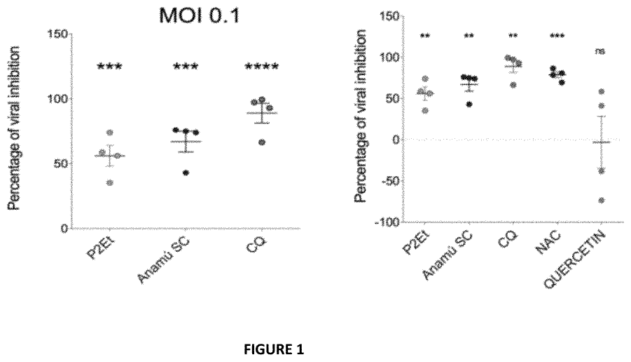

Culture supernatants were collected after infection and used to titrate the amount of infectious virus released. Using a MOI of 0.1, inhibition of the production of infectious viral particles is observed with P2Et treatment (FIG. 1). Chloroquine treatment was used as a control for inhibition of viral particles production, N-Acetylcysteine (NAC) was used as antioxidant, anamu as pro-oxidant and quercetin as polyphenol. Statistical analysis of the significance between two groups was calculating using two-tailed unpaired and paired Student's t test for most of the statistical analyses and a p value of <0.05 was considered statistically significant. The specific statistical test results are indicated in each figure: *p<0.05; **p<0.01; ***p<0.001; ****p<0.001. Statistical analyses were performed in Graph Pad Prism version 8.0.

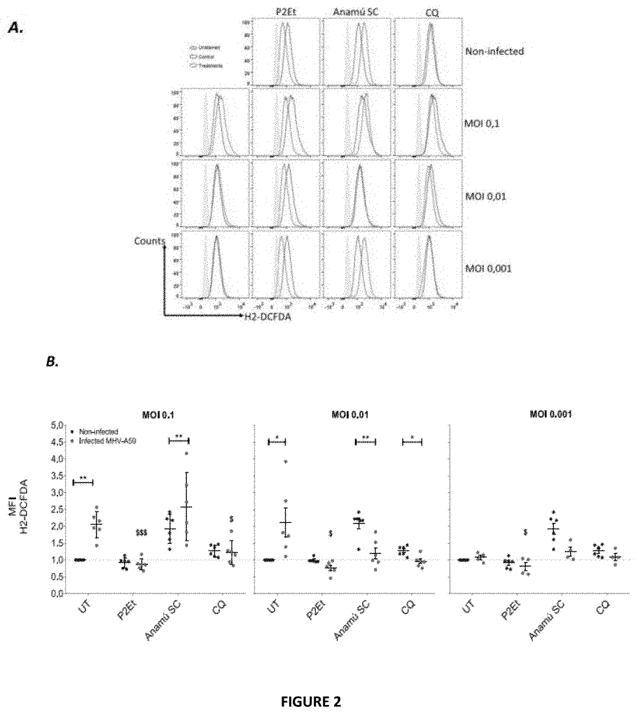

To get an insight into P2Et's cellular mechanisms involved in the virus inhibition, the ability of the virus to induce oxidative stress in the absence or presence of P2Et was evaluated. Thus, the treatment was maintained during adsorption and viral replication. As expected, fibroblasts L929 virus infection induced an increase in ROS—evaluated with the H2-DFCDA probe—in a manner dependent on the amount of virus used, whereas P2Et extract decreased L929 intracellular ROS despite virus infection (FIG. 2).

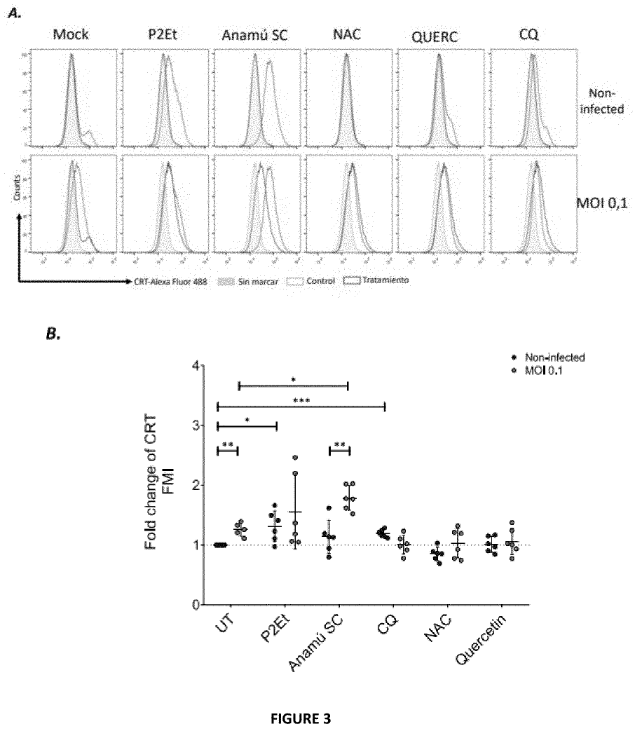

Previously, we have shown that standardized P2Et extract induces immunogenic cell death (ICD), here the expression of calreticulin, a marker of ICD, was evaluated on the L929 cell membrane infected cells. The findings showed that MHV-A59 virus infection induced the expression of calreticulin on the L929 cell surface, whereas chloroquine, NAC, and quercetin decreased the expression of calreticulin induced by viral infection. Conversely, the anamu treatment increased marker expression and P2Et expression held up calreticulin expression (FIG. 3), a pharmacological double target, since P2Et treatment further to induce viral inhibition replication, additionally, promote an immune response against the virus.

### Example 2. Anti-Inflammatory and Lung Fibrosis Reduction in the Bleomycin Model Induced by the Standardized P2Et Extract of the Disclosure

Bleomycin animal model of pulmonary fibrosis, mainly characterized by fibrotic response following acute lung injury, has been used as preclinical model for antifibrotic therapy, one of the major causes of mortality in COVID-19 (George, P. M., et al., 2020). C57BL6 mice were inoculated with bleomycin and the next day they were treated or not with P2Et extract (5 animals per group), every 3 days. On day 8, all animals were sacrificed, and the lungs and serum were obtained to analyze the innate, adaptive, myeloid immune compartment and inflammatory intracellular cytokines by flow cytometry and RT-PCT.

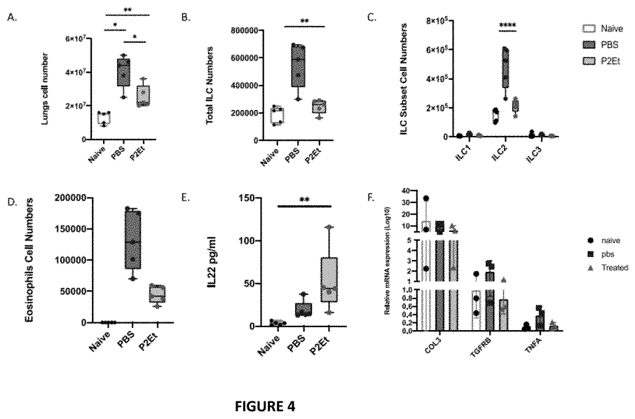

Animals that were subjected to airway stress with bleomycin showed an increase in pulmonary infiltrate 8 days post-treatment, which can be deleterious to the lung. Treatment with P2Et extract reduces the inflammatory infiltrate (FIG. 4A) linked to less innate infiltrated cells of the lungs (FIG. 4B), particularly ILC2 subset (FIG. 4C). These results suggest that P2Et treatment inhibits lung ILC2 recruitment or expansion, which is associated to poor prognosis during pulmonary fibrosis evolve.

In addition, animals treated with P2Et have significantly less infiltration of eosinophils into the lungs (FIG. 4D), which are considered as inflammation markers that can induce tissue damage. Likewise, ILC2 cells are IL-5 major producers, a soluble factor involved in eosinophil recruitment toward tissues. These results suggest that decrease in ILC2 subset due to P2Et extract treatment could affect the lungs eosinophils recruitment. In fact, the analysis of soluble factors evidenced a significant IL-22 increase in the P2Et treatment group, a cytokine produced by innate immune cells and linked to protective effect during pulmonary fibrosis development (FIG. 4E). Pulmonary fibrosis progression is characterized by collagen accumulation, response increase to TGFb and TNFa increase. These factors were evaluated in tissue samples and a significant decrease in the TGFb and TNFa receptor was observed (FIG. 4F). It is generally suggested that P2Et treatment inhibits or regulates the progression of bleomycin-induced fibrosis.

### EXAMPLE 3. Study to Evaluate the Efficacy and Safety of Standardized P2Et Extract for Patients with Moderate and Severe Coronavirus Disease 2019 (COVID-19)

The CS002-COVID19 protocol was a randomized, double-blind, placebo-controlled study that sought to demonstrate the efficacy and safety of treatment with P2Et in patients with a clinical diagnosis of COVID-19 infection by adding it to standard therapy. This study was carried out in two research centers, the San Ignacio University Hospital, and the Cardio VID Clinic, located in Bogota and Medellin, Colombia, respectively. Allocation to treatment groups was done using a random number generator. A total of 91 patients who met the inclusion criteria were enrolled and randomized; 44 patients were assigned to the P2Et experimental group and 46 to the placebo group. Patients assigned to the experimental group received 500 mg of standardized P2Et extract daily distributed into two post-prandial doses of 250 mg every 12 hours for 14 days. Patients assigned to the control group received a postprandial placebo capsule every 12 hours for 14 days.

Statistical analysis. Log-rank test was used for the primary endpoint and the Chi-Square test for the secondary endpoints. For normal quantitative variables like the leukocyte subpopulations in PB (absolute, percentage and delta), the t-test was used for the comparison of averages; for those without normal distribution, the Mann Whitney U-test was used. A p value of <0.05* was considered statistically significant. Data from ILCs analysis was analyzed using GraphPad Prism v9.3.1 for Mac OS X (GraphPad Software, La Jolla Calif. United States, www.graphpad.com). Statistical analysis of the significance between two groups was calculated using the Mann-Whitney U test. Differences among subject groups were evaluated using Kruskal-Wallis and Dunn's posttest for multiple comparisons. For all cases, the differences were considered statistically significant when p<0.05.

### Example 3.1 Adverse Events

A total of 75 adverse events occurred, 47 of them were classified as grade 1, 24 as grade 2, 2 as grade 4 and 2 as grade 5. There were 2 deaths in this group of patients that were not related to the drug. Unlike phase I, only 1 patient had grade 1 epigastric pain in the P2Et group, and 2 patients had grade 1 diarrhea. There were no significant risks from the safety data received during the reporting period. No unexpected safety signs were observed with the use of P2Et extract in healthy volunteers or in patients with COVID19.

### Example 3.2 Clinical Efficacy

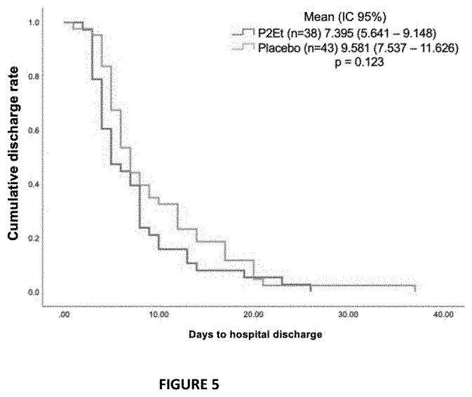

Regarding the primary endpoint, the median length of hospitalization in the P2Et group was 7.395 days (IC 95% 5.641-9.148) vs. placebo group with a median of 9.581 (IC 95% 7.537-11.626) (p value: 0.123) (FIG. 5).

### Example 3.3 Modulation of Pro-Inflammatory Cytokines after P2Et Extract Treatment

Peripheral blood samples from patients were collected on 1 day and day 14. All patients provided written informed consent to participate in the study. Peripheral blood mononuclear cells (PBMCs) were isolated by density-gradient centrifugation using Ficoll-Paque™ PREMIUM (GE Healthcare, Chicago, Ill., USA). A total of 1×107 cells were cryopreserved in liquid nitrogen in freezing media (RPMI-1640 50%, FBS 40% and 10% DMSO) until use for flow cytometry characterization. Serum from patients were collected and conserved at −80° C. for cytokine analyzes.

Cytokine measurement (G-CSF, IFN-γ, IL-10, IL-13, IL-15, IL-2, IL1-RA, IL-4, IL-5, IL-6, IL-7, IL8, IP-10, MCP-1, MIP1-α, TNF-α, MCP-2, TSLP, IL-33, NTproBNP, IL-14, IL-18, IL-22 and IL17E) was performed in serum of COVID-19 patients (days 1 and 14) and healthy donors (n=10) using a different commercially available multiplex immunoassay (MILLIPLEX MAP Human Cytokine/Chemokine Magnetic Bead Panel Immunology and Panel II Immunology Multiplex Assay; MILLIPLEX MAP Human Cardiovascular Disease (CVD) Magnetic Bead Panel 1, Cardiovascular Multiplex Assay; MILLIPLEX MAP Human TH17 Magnetic Bead Panel-Immunology Multiplex Assay; MILLIPLEX MAP Human Cytokine/Chemokine Magnetic Bead Panel IV; MILLIPLEX Human Cytokine/Chemokine/Growth Factor Panel A-Immunology Multiplex Assay, Millipore, Burlington, Mass., USA). The assay was performed in 96-well plates according to the manufacturer's instructions and the results were expressed in pg/mL. Serum samples (25 μL) were added into the wells with 25 μL of assay buffer, followed by the addition of matrix solution. After mixing, 25 μL of beads were added to the wells, and plate was incubated overnight at 4° C. with shaking. After incubation, fluid was removed, and plate was washed. After addition of 25-μL detection antibodies, plate was incubated for 1 h at room temperature with shaking. Then, 25 μL of streptavidin-phycoerythrin was added to each well, and plate was further incubated for 30 min at room temperature with shaking. Fluid was removed, and plate was washed. Then, 150 μL of sheath fluid was added. After resuspension for 5 min, the median fluorescent intensities were determined on a MAGPIX® System. Cytokines concentrations were calculated through the five-parameter logistic curve-fitting method using the median fluorescence intensity.

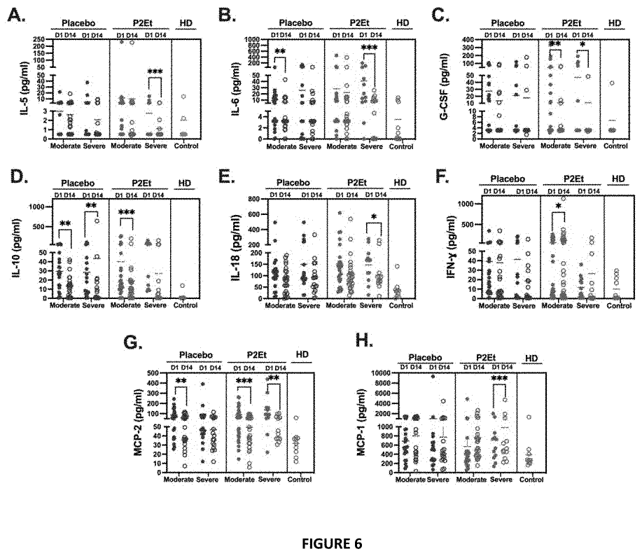

A down-regulation of pro-inflammatory cytokines such as G-CSF, IL-13, IL-15, IL-12, IL-6, IP10, MCP-1, MCP-2, IL-18 and IL-17 were observed in both groups. However, in the group of patients with severe COVID-19 P2Et, a statistically significant reduction was observed at day 14 in the following cytokines: IL-5 (FIG. 6A), IL-6 (FIG. 6B), G-CSF (FIG. 6C), IL-10 (FIG. 6D), IL-18 (FIG. 6E) and MCP-2 (FIG. 6G). Likewise, in the group of patients with moderate COVID-19 P2Et, a statistically significant decrease in G-CSF (FIG. 6C) and MCP-2 (FIG. 6G) as well as an IFN-γ significant increase were observed (FIG. 6F). Additionally, an MCP-1 increase was established in the group of patients with severe COVID-19 P2Et (FIG. 6H). There was no difference between P2Et treatment and placebo in other cytokines like: TNF-α, IL-2, IL-4, IL-7, IL-8, IL1-RA, IL-9, IL-13, IL-14, IL-15, IL-17, IL-18, IL-22, IP-10, MIP-α, TSLP, NT-PRO-BNP.

### Example 3.4 Immune Cells Modulation after Placebo or P2Et Treatment

PBMCs were thawed at 37° C. and washed with PBS 1X. Cells were stained with the green viability dye for 30 min at 4° C. in the dark, then washed with FACS buffer and stained with the antibody mix for 30 min at 4° C. in the dark. The antibody mix was composed as follow: FITC Lineage (CD3, CD4, CD14, CD15, CD19, CD20, CD33, CD34, CD203c, FceRIa), CD8 PE, CD127 PE-Dazzle, NKp46 PercP-Cy5.5, KLRG1 PE-Cy7, CD294 BV421, CD94 BV786, CD25 BV510, CD56 BV570, CD117 BV605, CD69 BV650, PD1 BV711, CD278 APC, CD16 Alexa Fluor 700 and CD27-APCeF780. Then, the cells were acquired by flow cytometry using Cytek Aurora (Cytek Biosciences, Fremont, Calif.), and the results were subsequently analyzed using SpectraFlo® software V 3.0 (Cytek Biosciences, Fremont, Calif.), and FlowJo 10.8.1 Software (Tree star, Ashland, Oreg.).

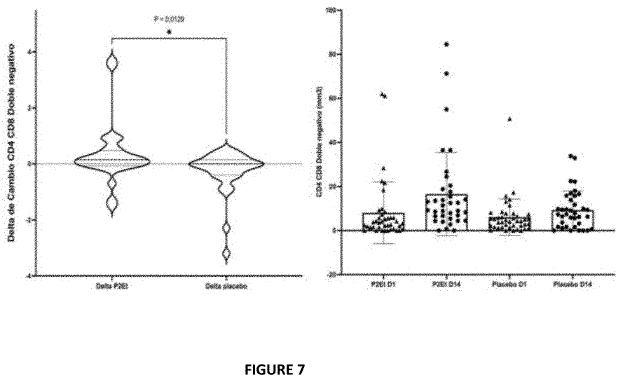

A decrease in the absolute values of CD3+, CD3+CD4+, CD3+CD8+, CD3+CD4+ CD8+ and CD3+CD4−CD8− was observed in non-survivors of COVID-19, mainly in men [12]. Regarding the data, P2Et extract seems to exert a protective role in COVID-19 trough the increase of the delta of CD3+CD4−CD8− T lymphocytes in severe illness patients and the frequency and relative values of CD3+CD4−CD8− cells (Table 1 & FIG. 7); this effect is particularly evident in the female population.

TABLE 1

| | | Placebo (n = 34) | | P2Et (n = 33) | | |
|---|---|---|---|---|---|---|
| Parameter | | D 1 | D 14 | D 1 | D 14 | p |
| CD3+ | Absolute (mm3) | 580.89 | 1164.02 | 765.09 | 1249.18 | |
| | % | 62.0455 | 64.6333 | 59.2818 | 67.8879 | |
| | Delta % | 2.59 | | 8.61 | | 0.027 |
| CD4+ | Absolute (mm3) | 304.50 | 629.33 | 414.02 | 698.09 | |
| | % | 32.0758 | 34.1727 | 32.8121 | 37.5697 | |
| | Delta % | 2.10 | | 4.76 | | 0.392 |
| CD8+ | Absolute (mm3) | 242.63 | 484.84 | 305.01 | 473.56 | |
| | % | 26.3384 | 26.6153 | 22.9606 | 26.4727 | |
| | Delta % | 0.28 | | 3.51 | | 0.081 |
| Relation CD4 CD8 | Absolute (mm3) | 1.46 | 1.49 | 1.75 | 1.71 | |
| | % | 0.04 | | −0.03 | | 0.247 |
| CD4+ CD8+ | Absolute (mm3) | 8.91 | 14.10 | 16.96 | 30.26 | |
| | % | 1.0152 | 0.7697 | 1.3364 | 1.4394 | |
| | Delta % | −0.25 | | 0.10 | | 0.190 |
| CD4− CD8− | Absolute (mm3) | 6.63 | 7.68 | 8.29 | 15.03 | |
| | % | 0.6879 | 0.4397 | 0.5816 | 0.8970 | |
| | Delta % | −0.25 | | 0.32 | | 0.007 |
| Lymphocites T | Absolute (mm3) | 19.14 | 37.02 | 20.26 | 34.70 | |
| Gamma Delta | % | 1.9939 | 2.0788 | 1.5303 | 1.6848 | |

| | | | |
|---|---|---|---|
| Delta % | 0.08 | 0.15 | 0.312 |

### Example 3.5 ILC, ILC2 and NK Populations in COVID Patients after Treatment with P2Et Extract or Placebo

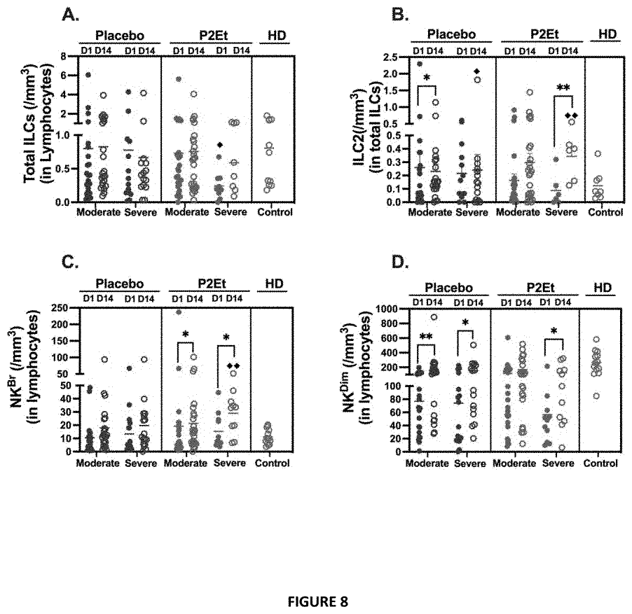

It has been reported that total ILCs count decreased in a significant manner during COVID-19 disease (Iannetta, M., et al., 2021). However, no difference between P2Et treatment and placebo groups were observed. At day 1, there was a significant decrease in total ILCs count between patients with severe COVID-19 P2Et group and healthy donors (FIG. 8A). At day 14, there was a significant decrease in ILC2 subset in the placebo group of patients with moderate COVID-19 (FIG. 8B) and significant ILC2 peripheral subset recovery in the P2Et group of patients with severe COVID-19 (FIG. 8B). This peripheral cell population can exert a protective effect correlated with respiratory parameters improvement.

After 14 days of treatment, a significant NKBr recovery—even reaching higher levels than healthy donors—was observed in the P2Et group of patients with moderate and severe COVID-19, which suggests better peripheral immune response to COVID-19 (FIG. 8C). Although there was evidence of a NKDim recovery in the placebo group (moderate and severe), a higher subset recovery was evidenced in the P2Et group of patients with severe COVID-19 (FIG. 8D).

1. Therapeutical composition for the treatment or preventing viral infections in particular SARS-CoV-2 infections comprising a standardized P2Et extract enriched in polyphenol compounds from Caesalpinia spinosa.

2. A therapeutical composition enriched in polyphenol compounds according to claim 1 characterized in that standardized P2Et extract may comprise one or more compounds selected from galloyl quinic acid and gallic acid derivatives.

3. A therapeutical composition enriched in polyphenol compounds from galloyl quinic acid according to claim 1, characterized in that the standardized P2Et extract comprises between 70% and 100%, relative to the total weight of the composition, from one or more compounds selected from n-O-galloylquinic acid, di-O-galloylquinic acid, tri-O-galloylquinic acid, tetra-O-galloylquinic acid, n-O-galloylquinic acid methyl ester, n-O-galloylquinic acid ethyl ester, di-O-galloyl quinic methyl ester, di-O-galloyl quinic ethyl ester, tri-O-galloyl quinic methyl ester, tri-O-galloyl quinic ethyl ester, di-O-galloyl quinic methyl gallate ester, tri-O-galloyl quinic methyl gallate ester, di-O-galloyl quinic ethyl gallate ester, tri-O-galloyl quinic ethyl gallate ester, n-O-galloyl(digalloyl)quinic acid, n-O-galloyl(galloyl)quinic acid, di-O-galloyl(digalloyl)quinic acid or di-O-galloyl(galloyl)quinic acid.

4. A therapeutical composition enriched in polyphenol compounds from galloyl quinic acid according to claim 1, characterized in that the standardized P2Et extract comprises:
(i) between 70% and 95%, relative to the total weight of the composition, of one or more compounds selected from:
n-O-galloylquinic acid, di-O-galloylquinic acid, tri-O-galloylquinic acid, tetra-O-galloylquinic acid, n-O-galloylquinic acid methyl ester, n-O-galloylquinic acid ethyl ester, di-O-galloyl quinic methyl ester, di-O-galloyl quinic ethyl ester, tri-O-galloyl quinic methyl ester, tri-O-galloyl quinic ethyl ester, di-O-galloyl quinic methyl gallate ester, tri-O-galloyl quinic methyl gallate ester, di-O-galloyl quinic ethyl gallate ester, tri-O-galloyl quinic ethyl gallate ester, n-O-galloyl(digalloyl)quinic acid, n-O-galloyl(galloyl)quinic acid, di-O-galloyl(digalloyl)quinic acid or di-O-galloyl(galloyl)quinic acid.
(ii) between 5% and 30%, relative to the total weight of the composition, of one or more compounds selected from: gallic acid, ethyl gallate or methyl gallate.

5. A therapeutical composition enriched in polyphenol compounds from galloyl quinic acid according to claim 1, characterized in that the standardized P2Et extract comprises: (i) between 70% and 95% relative to the total weight of one or more compounds selected from galloyl quinic acid and its derivatives and (ii) between 5% and 30%, relative to the total weight of one or more compounds from gallic acid and its derivatives to down-regulate pro-inflammatory cytokines and increase CD3+TL and CD3+CD4−CD8− T Lymphocytes subsets when is administered in a subject suffering from SARS-CoV-2 infections.

6. A therapeutical composition enriched in polyphenol compounds from galloyl quinic acid according to claim 1, wherein the standardized P2Et extract is incorporated into the drug product in a dosage form formulated with one or more pharmaceutically acceptable excipients for oral administration in solid or liquid pharmaceutical forms as tablets, enteric or conventional capsules, buccal forms and oral liquids, suspensions or solutions, for topical administration in heterodispersed W/O or O/W forms as creams, gels and ointments and for parenteral or rectal administration.

7. A therapeutical composition enriched in polyphenol compounds from galloyl quinic acid according to claim 1, wherein the standardized P2Et extract is obtained from C. spinosa pods extracted with ethanol 96% (DER 20-33,3:1) and subsequent fractionation step with ethyl acetate.

8. A method for treating or preventing SARS-CoV-2 infections in a human in need thereof comprising administering a therapeutically effective amount of a pharmaceutical composition containing one or more galloyl quinic acid and gallic acid derivatives from a standardized P2Et extract and pharmaceutically acceptable excipients according to claim 1.

9. The method for treating or preventing SARS-CoV-2 infections according to claim 8, wherein the therapeutically effective amount is from about 0.5 to about 50 mg/kg/day.

10. The method for treating or preventing SARS-CoV-2 infections according to claim 8, comprising administering to the human a daily dose from about 0.4 mg to about 4000 mg via oral route for 1 to 28 days, preferably from about 20 mg to about 2000 mg via oral route for 1 to 28 days.

11. A method to facilitate normalization of the cytokine profile, CD3+TL and CD3+CD4−CD8− T Lymphocytes subsets in a subject suffering from SARS-CoV-2 infections comprising administering to the subject a pharmaceutical composition containing a therapeutically effective amount of galloyl quinic acid and gallic acid derivatives from a standardized P2Et extract and pharmaceutically acceptable excipients according to claim 1.

12. The method of claim 11, wherein the therapeutically effective amount is from about 0.5 to about 50 mg/kg/day.

13. A method to inhibit the production of coronavirus particles and reducing pulmonary fibrosis markers, comprising administering to the subject a pharmaceutical composition containing a therapeutically effective amount of galloyl quinic acid and gallic acid derivatives from a standardized P2Et extract and pharmaceutically acceptable excipients according to claim 1.

14. The method of claim 13, wherein the therapeutically effective amount is from about 0.4 mg to about 4000 mg, preferably from about 20 mg to about 2000 mg for 1 to 28 days.

15. Use of a composition for treating or preventing SARS-CoV-2 infections comprising a standardized P2Et extract from pods of Caesalpinia spinosa wherein the standardized P2Et extract is incorporated into the drug product and comprising one or more compounds selected from galloyl quinic acid and gallic acid derivatives.

16. Use of a standardized P2Et extract from pods of Caesalpinia spinosa for manufacturing a composition for treating or preventing SARS-CoV-2 infections according to claim 15, comprising one or more compounds selected from galloyl quinic acid and gallic acid derivatives.
